(19) United States
(12) Patent Application Publication
(10) Pub. No.: US 2024/0335593 A1
(43) Pub. Date: Oct. 10, 2024

(54) GENERATING ALLOGENIC ENDOTHELIAL CELL-SEEDED VASCULAR GRAFTS AND METHODS OF USE THEREOF

(71) Applicant: Wisconsin Alumni Research Foundation, Madison, WI (US)

(72) Inventors: James THOMSON, Madison, WI (US); Jue Zhang, Madison, WI (US); John Maufort, Madison, WI (US)

(21) Appl. No.: 18/297,417

(22) Filed: Apr. 7, 2023

Related U.S. Application Data
(60) Provisional application No. 63/328,421, filed on Apr. 7, 2022.

(51) Int. Cl. A61L 27/50; A61L 27/38; C12N 5/071

(52) U.S. Cl. CPC A61L 27/507; A61L 27/3808; C12N 5/0692; A61L 2430/34

(57) ABSTRACT

Provided herein are allogeneic human endothelial cell-seeded polymeric vascular grafts suitable for replacing or bypassing natural blood vessels and exhibiting increased long term patency rates and reduced leukocyte adhesion relative to grafts comprising venous endothelial cells. Methods for generating the human endothelial cell-seeded vascular grafts and therapeutic uses of the same are also described.

This application claims priority to U.S. provisional application No. 63/328,421, filed Apr. 7, 2022, the disclosure of which is incorporated by reference in its entirety herein.

This invention was made with government support under HL134655 awarded by the National Institutes of Health. The government has certain rights in the invention.

### FIELD OF THE INVENTION

This invention relates to improved polymeric vascular grafts comprising endothelial cells that are scalable, available as on-demand products, and suitable for multiple patients.

### BACKGROUND

A frequent procedure in cardiovascular surgery is to replace or bypass a blood vessel in order to provide more adequate flow of blood to downstream tissues. In coronary artery bypass operations, arterial grafts have much better long term patency rates than venous grafts. If the operation requires multiple grafts, however, veins are often used because the patient lacks suitable additional arterial grafts. In bypass operations to treat peripheral artery disease, the arteries to be bypassed are generally so large that no suitable arterial grafts are available. Again, vein grafts are often used in spite of the comparatively high long-term rate of occlusion.

Many significant disadvantages are associated with transplantation of a patient's own vessels for bypass operations. The time required to excise the vessel and prepare it for transplant increases the patient's exposure to anesthesia, increases the chance of postoperative infection, and increases the cost of the procedure. For many patients, suitable arteries or veins are simply not available for grafting due to vascular disease or prior surgeries.

Because of the limitations of using a patient's own vessels as grafts, there have been numerous attempts at making vascular grafts from synthetic materials. While synthetic grafts generally work well for treatments involving the largest diameter vessels (e.g., the aorta), long term patency rates for synthetic grafts decrease as vessel size decreases. For peripheral artery disease of the leg, artificial grafts are used as a last resort when a suitable vein graft is not available, because artificial grafts in this location occlude at a higher rate than vein grafts. For bypass operations involving even smaller cardiac arteries (˜3-5 mm diameter vessels), synthetic grafts fail at such a high rate that they are not currently used.

One group has found that by lining a synthetic ePTFE material with a patient's own venous endothelial cells, they could improve long term patency rates of ePTFE grafts in peripheral arterial disease to approximately equal the long term patency rates of venous grafts (Deutsch et al., 1999). This procedure, however, requires harvesting a patient's vein in a separate surgery, expanding the venous endothelial cells in culture, lining the ePTFE tube with the venous endothelial cells, maturing the cells on the ePTFE during extended culture, and then finally transplanting the ePTFE/venous endothelial cell graft back to the patient. The entire procedure from vein harvest to transplant takes about a month (Deutsch et al., 2009). Thus, the procedure is expensive and slow: About ⅓ of the patients with peripheral artery disease have an acute need for intervention and cannot wait 30 days—the time necessary for production of the ePTFE/autologous venous endothelial cell graft. And because of immune rejection, this approach can only treat an individual patient and cannot be scaled up for the treatment of multiple patients. Finally, although the improvement in patency rates of venous endothelial cell-lined ePTFE grafts over standard ePTFE grafts was impressive, those rates still only approach the long term patency rates of vein grafts, not arterial grafts.

In addition, according to AHA update 2022, an estimated 371,000 in-patient coronary artery bypass procedures were performed for in-patients in the United States in 2014 (AHA update 2022). Autologous arteries or veins are primarily used in small-diameter blood vessel bypass surgery. However, autologous grafts require invasive harvesting, and some patents don't have suitable blood vessel grafts. Synthetic vascular grafts demonstrate long-term patency for large and medium diameter vessels, but generally fail for small diameter (less than 6 mm) vessels.

Accordingly, there remains a need in the art for improved polymeric vascular grafts comprising endothelial cells that are scalable, available as on-demand products, and suitable for multiple patients.

### SUMMARY OF THE DISCLOSURE

Provided herein are methods for treating blood vessel defects in a subject in need thereof comprising implanting a vascular graft in the subject, wherein the vascular graft comprises (a) a polymeric substrate at least partially coated by an endothelial cell attachment agent and (b) human endothelial cells adhered to said coated polymeric substrate. The vascular grafts provided herein can be used to treat blood vessel defects arising from trauma or vascular disease and are implanted inter alia by anastomosis.

Further provided herein are methods for implanting a vascular graft in a subject in need thereof, wherein the vascular graft comprises (a) a polymeric substrate at least partially coated by an endothelial cell attachment agent and (b) human endothelial cells adhered to said coated polymeric substrate.

Provided herein is composition of a vascular graft comprising (a) a polymeric substrate at least partially coated by an endothelial cell attachment agent and (b) human endothelial cells adhered to said coated polymeric substrate.

In particular embodiments of the vascular grafts provided herein the human endothelial cells are arterial endothelial cells. In other particular embodiment, the vascular graft comprises venous endothelial cells.

The endothelial cells as provided herein are not autologous cells, and in certain embodiments are allogeneic cells. The endothelial cells as provided as part of the vascular graph are advantageous, inter alia, because they are not necessarily major histocompatibility complex (MHC) matched to the subject, nor have they necessarily been genetically modified to reduce rejection by the immune system. These features of the vascular graft provided herein are advantageous as being contrary to conventional wisdom that graft should be autologous, MHC-matched, or genetically modified to reduce rejection by the immune system, all of which have been found as disclosed herein to be unnecessary. Accordingly, the vascular graft produced and provided as disclosed herein have the advantage of being capable of being “off-the-shelf” products that do not need to be custom made for each individual in need thereof.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The following drawings form part of the present specification and are included to further demonstrate certain aspects of the compositions and methods provided herein. The invention can be better understood by reference to one or more of these drawings in combination with the detailed description of specific embodiments presented herein.

FIG. 1A shows flow cytometric analysis of the arterial endothelial cells (AEC) generated from rhesus embryonic stem cells used in Example 2. FIG. 1B shows immunostaining of rhesus AECs (rAEC) on vascular grafts. FIG. 1C shows a three-dimensional image of the vascular graft implanted in Example 2.

FIG. 2A shows a schematic of the rhesus model used in Example 2 where the vascular graft was transplanted into the rhesus femoral artery. FIG. 2B shows ultrasound images used to measure the flow rate of vascular grafts. FIG. 2C shows images of the middle part of the vascular grafts following implantation. The naked-graft was completely blocked by a blood clot (thrombosis), while the allogenic AEC-graft remained patent. FIG. 2D shows statistical data illustrating the patency of the grafts over time.

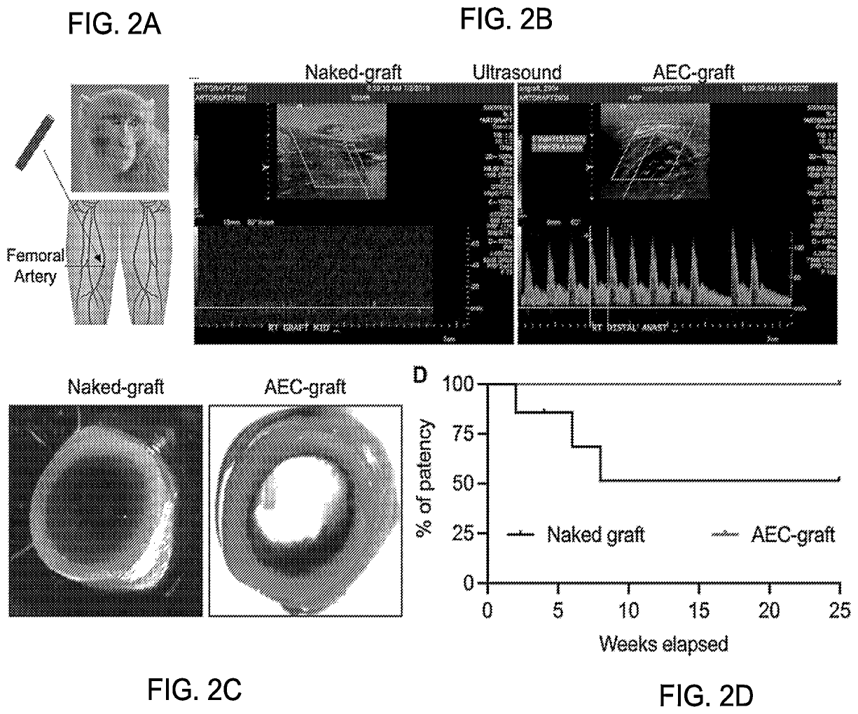

FIG. 3 shows the patency of double knockout of Beta-2-Microglobulin (B2M) and Class II transactivator (CIITA) AEC graft (DKO-AEC-graft) as compared to a naked-graft.

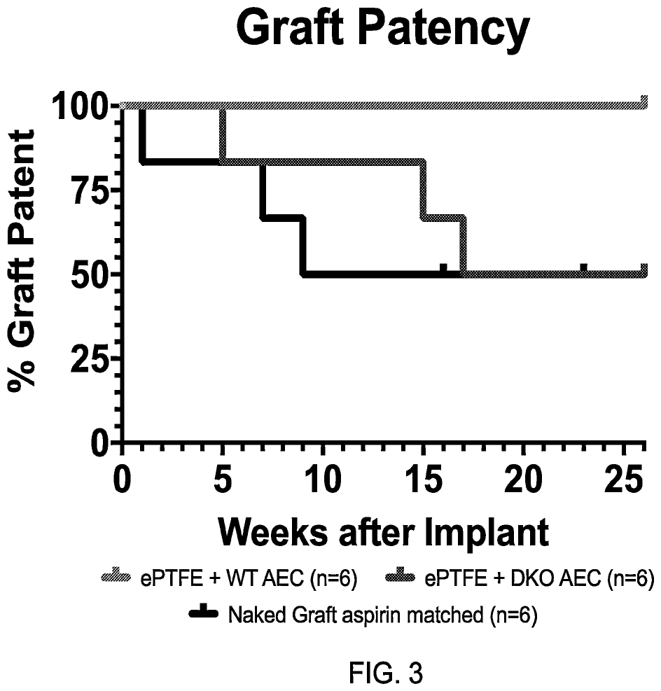

FIG. 4 is a bar graph showing the percent of donor cells remaining in vascular grafts produced and employed in vivo in a rhesus macaque model, where rh2833, rh 2837, ro5045, rh2500, r13005 are samples from MHC-WT grafts, and PuroR⁻ and Puro⁺ DNA are used as negative and positive control respectively as set forth herein below.

### DETAILED DESCRIPTION OF THE DISCLOSURE

Provided herein are method for treating a blood vessel defect in a subject in need thereof comprising implanting a vascular graft in a subject, wherein the vascular graft comprises (a) a polymeric substrate at least partially coated by an endothelial cell attachment agent and (b) human endothelial cells adhered to said coated polymeric substrate.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other component, feature, element, or step or integer, step, or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

All publications, including but not limited to patents and patent applications, cited in this specification are herein incorporated by reference as though set forth in their entirety in the present application.

### I. Compositions

In a first aspect, provided herein is a polymeric vascular graft comprising venous endothelial cells. In a second aspect, provided herein is a polymeric vascular graft comprising arterial endothelial cells. The graft can comprise or consist essentially of a polymeric substrate at least partially coated by an endothelial cell attachment agent, and endothelial cells (EC) adhered to coated polymeric substrate. As described herein, EC-seeded polymeric grafts of this disclosure exhibit increased patency relative to conventional polymeric vascular grafts. In some embodiments, human endothelial cells used with the polymeric vascular grafts of this disclosure are allogeneic. An allogeneic graft is produced using ECs, harvested from a donor other than the recipient.

In some embodiments, human endothelial cells used with the polymeric vascular grafts of this disclosure are not non-major histocompatibility complex (MHC) matched. Despite an apprehension that, when a human transplant is performed, HLA (human leukocyte antigens) molecules from a donor could be recognized by the recipient's immune system triggering an alloimmune response, as provided herein vascular grafts of the invention can be provided that are allogenic. Further and specifically the vascular grafts as provided herein are not donor and recipient matched for MHC antigens whose endothelium will be partially or completely repopulated with the host endothelial cells. Instead, as set forth herein the inventors surprisingly found that using non-MHC matched ECs cells resulted in superior patency of the vascular graft.

Further, genetic modification (such as modifying a cell to express certain cell surface markers such as proteins encoded by either the class II or both the class I and the class II major histocompatibility complex genes) is not desirable for human endothelial cells used with the polymeric vascular grafts of this disclosure as a way reduce rejection by the immune system. In this way, the cells are not genetically engineered to mitigate immune response and thus, are immunogenic to the recipient. Reducing immunological rejection to evade an immune response increases the chance of tumorigenesis. Accordingly, grafts provided by the invention have a lower risk and frequency of tumorigenesis.

As used herein, the terms “graft” and “vascular graft” are used interchangeably herein and refer to any conduit or portion thereof intended as a prosthetic device for conveying fluid (e.g., blood) and therefore having a fluid-contacting (i.e., “luminal”) surface. While it is intended primarily as a tubular form, the graft can also be a partial tube or sheet material useful for patching portions of the circumference of living blood vessels (these materials are generally referred to as cardiovascular patches). Likewise, the term vascular graft includes intraluminal grafts for use within living blood vessels. For example, vascular grafts provided herein can be used as a sheath or other covering on the exterior surface, luminal surface, or both luminal and exterior surfaces of an implantable vascular stent.

As used herein, the term “EC seeded” and grammatical variations thereof refer to a substrate upon which endothelial cells are provided including arterial and venous endothelial cells. Preferably, the term refers to polymeric vascular grafts bearing endothelial cells (e.g., human ECs) and an endothelial cell adhesion agent, whereby the seeded polymeric vascular graft is suitable for implantation into a subject.

As used herein, the term “patency” refers to the degree of openness of a tube, such as a blood vessel or vascular graft. A vascular graft having 100% patency is free of any blockage or obstruction. As the degree of blockage or obstruction increases, patency of the vessel or vascular graft decreases. In this manner, patency of a vessel or vascular graft is a proxy for graft success or failure. In some instances, patency is assessed at a particular time point including, without limitation, patency of a vascular graft days, weeks, months, or years following implantation. Preferably, polymeric vascular grafts of this disclosure exhibit increased long-term patency rates relative to conventional polymeric vascular grafts. As used herein, “long-term patency” means a vessel or graft remains patent in a physiological environment for more than 1 year, preferably more than 3 years, more preferably more than 5 years, and most preferably 10 years or more following implantation. In some instances, EC-seeded polymeric vascular grafts of this disclosure exhibit patency that matches and, preferably, outperforms autologous grafts.

### A. Materials for Compositions

Polymeric vascular grafts provided herein comprise a polymeric substrate at least partially coated by an endothelial cell attachment agent, and human endothelial cells adhered to said coated polymeric substrate. Suitable polymeric materials for the vascular grafts provided herein include, without limitation, poly vinyl chloride (PVC), PGA (poly glycolic acid), PLA (poly lactic acid), PCL (poly caprolactone), polylactic-co-glycolic acid (PLGA), polyurethane, polydioxanone, polyethylene terephthalate (Dacron®), polyethylene, and fluoropolymers such as tetrafluoroethylene (TFE), polytetrafluoroethylene (PTFE), and expanded polytetrafluoroethylene (ePTFE). PTFE is a homopolymer of tetrafluoroethylene (TFE). When PTFE is stretched and expanded into ePTFE, the polymeric material is particularly suitable for vascular applications as it exhibits low thrombogenicity and can be extruded as a tube, sheet, or other suitable graft shape. In some embodiments, the polymeric substrate is a GORE-TEX®; vascular graft.

In some embodiments, biological materials are suitable for polymeric substrates of this disclosure. For example, polymeric substrates can comprise biological materials including, without limitation, silk, a decellularized construct (such as decellularized artery, vein, or small intestine), an extracelluar matrix protein-based scaffold (such as collagen, MATRIGEL™ fibrin, elastin), hyaluronic acid, chitosan, polyhydroxyalkanoates.

In some embodiments, polymeric substrates used for the vascular grafts provided herein are biocompatible, which means that the substrate material will not cause adverse reactions when implanted or placed in contact with the body.

In some embodiments, the polymeric substrate is a porous substrate. Without being bound to any mode of action or theory, it is believed that pores in the polymeric vascular grafts allow for recruitment and integration of host cells into the graft. For example, ePTFE exhibits high porosity and comprises a matrix of nodes and fibrils. The fibrils are thin connections between the nodes and are submicron in size. Thin fibrils are used to create more tortuosity and surface area in a membrane, impacting the filtration efficiency. In some embodiments, the geometry of fibrils and nodes in the membrane is modified (e.g., increasing or decreasing pore size(s), pore distribution) to customize the material's functionality. In some embodiments, an intermodal distance of about 7 μm to about 20 μm is preferred. In some embodiments, the polymeric substrate is microporous, meaning that pores of the porous substrate have micrometer scale sizes. Preferably, pore sizes of suitable polymeric substrates are within, and preferably cover, the range of 2 micron to 80 micron, preferably in the range from 3 micron to 40 micron, most preferably in the range from 5 micron to 35 micron, in particular around 30 micron.

In some embodiments, the disclosed vascular grafts are substantially tubular in shape with a round or substantially round cross-section.

The disclosed vascular grafts are substantially tubular in shape with a round or substantially round cross-section. In some embodiments, the tubular structure has a wall thickness of about 200 μm to about 500 μm (e.g., about 200, 250, 300, 350, 400, 450, 500 μm). In other embodiments, the polymeric substrate is a planar sheet or “patch” of polymeric material. In such embodiments, the thickness can vary widely from about 0.2 mm to about 1.0 mm or more.

The various dimensions of a polymeric vascular graft of this disclosure can vary according to the desired use. In principle, the dimensions will be similar to those of the host tissue in which the vascular graft is being used to replace. Generally, tubular grafts have a lumen extending throughout the length of the graft. The lumen of a vascular graft provided herein can be of any appropriate diameter that is suitable for the intended surgical use of the graft. For instance, average luminal dimensions of coronary arteries, including those having a higher incidence of occlusions (anterior interventricular artery, right coronary artery, circumflex artery) are well described in the literature. In some embodiments, the polymeric substrate has an inner diameter of about 0.5 mm to about 10 mm (e.g., about 0.5, 0.75, 1, 2, 3, 4, 5, 6, 7, 8, 9, 10 mm). The vascular grafts can be of any appropriate length that is suitable for the intended surgical use of the graft. Typically, the graft should be slightly longer than the length of artery or vein that is to be replaced.

In some embodiments, polymeric substrates used for the vascular grafts provided herein are hydrophobic membranes, meaning that they resist wetting by fluids (e.g., biological fluids) and are not chemically changed or degraded by biological fluids. In some embodiments, the hydrophobic membrane is impermeable to fluids but permit gas flow through the membrane.

In some embodiments, the polymeric substrate is at least partially coated by an endothelial cell adhesion agent. Endothelial cell adhesion agents useful for the vascular grafts of this disclosure include, without limitation, dopamine, fibrin, RGD (Arg-Gly-Asp)-peptides, and extracellular matrix proteins such as vitronectin and laminin, or mixtures of two or more adhesion agents. Conventionally, a network of blood coagulation protein fibrin (Fb) (sometimes referred to as “fibrin adhesive” or “fibrin glue”) has been used to coat vascular grafts. Preferably, the endothelial cell adhesion agent is non-immunogenic. For example, an endothelial cell adhesion agent preferably does not comprise any component derived from a non-human animal and is, thus, free of xenogeneic material (“xeno-free”). As used herein, the terms “free of xenogeneic materials” and “xeno-free” are used interchangeably and refer to materials (e.g., cell substrate, culture medium) or cell culture conditions that are free of any cell or cell product of species other than that of the cultured cell or the recipient of the materials.

In some embodiments, the endothelial cell adhesion agent comprises dopamine, where the dopamine is dissolved in a buffered solution at a concentration of about 0.1 mg/ml to about 20 mg/ml (e.g., about 0.1, 0.2, 0.3, 0.4, 0.5, 0.6, 0.7, 0.8, 0.9, 1, 1.5, 2, 2.5, 3, 4, 5, 6, 7, 8, 9, 10, 12, 14, 16, 18, 20 mg/ml dopamine). Where the endothelial cell adhesion agent comprises RGD peptides, the peptides can be provided in a buffered solution at a concentration of about 0.5 mM to about 10 mM (e.g., about 0.5, 0.6, 0.7, 0.8, 0.9, 1, 2, 3, 4, 5, 6, 7, 8, 9, 10 mM RGD peptide). In some embodiments, the endothelial cell adhesion agent comprises vitronectin. In some embodiments, vitronectin is provided in a buffered solution at a concentration of about 1 μg/ml to about 50 μg/ml (e.g., about 1, 2, 5, 10, 15, 20, 25, 30, 35, 40, 45, 50 μg/ml vitronectin). As used herein, the term “vitronectin” refers to a vitronectin polypeptide or fragment or peptide thereof, and encompasses recombinant vitronectin polypeptides and peptides (e.g., recombinant human vitronectin) and vitronectin polypeptide variants such as those described by U.S. Pat. No. 9,133,266, incorporated herein by reference as if set forth in its entirety. In some embodiments, the endothelial cell adhesion agent comprises laminin. In some embodiments, laminin is provided in a buffered solution at a concentration of about 1 μg/ml to about 50 g/ml (e.g., about 1, 2, 5, 10, 15, 20, 25, 30, 35, 40, 45, 50 μg/ml laminin). As used herein, the term “laminin” refers to a laminin polypeptide or fragment or peptide thereof, and encompasses recombinant laminin polypeptides and peptides (e.g., recombinant human laminin).

In some embodiments, the polymeric substrate can be partially or fully coated by or immersed in a solution comprising the endothelial cell adhesion agent for about 4 to about 24 hours. Coating by immersion in the endothelial cell adhesion agent solution can occur at any appropriate temperature including, without limitation, at 4° C., 25° C. (room temperature), or 37° C. Preferably, coated polymeric substrates are rinsed with distilled water or a buffered solution prior to use.

In some instances, it is advantageous to de-gas the polymeric substrate material prior to coating at least partially with an endothelial cell adhesion agent as demonstrated in U.S. patent application Publication No. US 20220296782, incorporated by reference in its entirety herein. As described in the Examples that follow, the inventors demonstrated reduced cell aggregate formation, improved cell density, and improved coating with endothelial adhesion agents when polymeric substrates were de-gassed before use. De-gassing can be performed by well-known methods in the art. As described in the Example, the polymeric substrate can be de-gassed in a series of acetone and ethanol washes. In some instances, de-gassing is performed as described but using an organic solvent in place of acetone. In other instances, high powered vacuum can be applied to the substrate to de-gas prior to use.

In some embodiments, endothelial cells are seeded onto coated (e.g., partially or fully coated), degassed polymeric substrates at a cell density of about 0.5×10⁶ cells/ml to about 3×10⁶ cells/ml. In some embodiments, ECs at a density of about 1×10⁶ to about 1.5×10⁶ cells/ml are seeded onto a prepared polymeric substrate.

ECs can be provided in any appropriate cell culture medium for seeding polymeric substrates. For example, ECs can be provided in a chemically defined cell culture medium that is xeno-free, serum-free, and albumin-free. As used herein, the terms “chemically defined culture conditions.” “fully defined, growth factor free culture conditions,” and “fully defined conditions” indicate that the identity and quantity of each medium ingredient is known and the identity and quantity of supportive surface is known. As used herein, “serum-free” means that a medium does not contain serum, or that it contains essentially no serum. For example, an essentially serum-free medium can contain less than about 1%, 0.9%, 0.8%, 0.7%, 0.6%, 0.5%, 0.4%, 0.3%, 0.2% or 0.1% serum. As used herein, the term “albumin-free” indicates that the culture medium used contains no added albumin in any form (such as in serum replacement), including without limitation Bovine Serum Albumin (BSA) or any form of recombinant albumin. Preferably, human ECs are seeded onto a polymeric substrate in a chemically defined cell culture medium that is free of any xenogeneic materials, that is to say free of any components derived from a non-human animal.

In some embodiments, seeding is performed by injecting a suspension of ECs into the lumen of a tubular vascular graft and placing the graft in a rotating device. In some embodiments, seeding is followed by maturation of the seeded substrate in culture flasks with fresh medium without rotation at any temperature suitable for cell growth such as, for example, at room temperature or preferably at 37° C. For example, seeding is, in some embodiments, followed by 2-3 days of maturation in culture flasks with fresh culture medium without rotation in a humid incubator at 37° C. In some embodiments, EC-seeded grafts are cultured under in the presence of 5% CO₂.

It can be appropriate, in some embodiments, to include a Rho-Kinase (ROCK) inhibitor in the cell culture medium for seeding polymeric substrates with ECs. Kinase inhibitors, such as ROCK inhibitors, are known to increase plating efficiency and viability of single cells and small aggregates of cells. See, e.g., US Patent Application Publication No. 2008/0171385, incorporated herein by reference as if set forth in its entirety; and Watanabe et al., 2007, “A ROCK inhibitor permits survival of dissociated human embryonic stem cells.” Nat. Biotechnol. 25:681-686. ROCK inhibitors suitable for use herein include, but are not limited to, (S)-(+)-2-methyl-1-[(4-methyl-5-isoquinolinyl) sulfonyl]homopiperazine dihydrochloride (informal name: H-1152), 1-(5-isoquinolinesulfonyl) piperazine hydrochloride (informal name: HA-100), 1-(5-isoquinolinesulfonyl)-2-methylpiperazine (informal name: H-7), 1-(5-isoquinolinesulfonyl)-3-methylpiperazine (informal name: iso H-7), N-2-(methylamino) ethyl-5-isoquinoline-sulfonamide dihydrochloride (informal name: H-8), N-(2-aminoethyl)-5-isoquinolinesulphonamide dihydrochloride (informal name: H-9), N-[2-p-bromo-cinnamylamino) ethyl]-5-isoquinolinesulfonamide dihydrochloride (informal name: H-89), N-(2-guanidinoethyl)-5-isoquinolinesulfonamide hydrochloride (informal name: HA-1004), 1-(5-isoquinolinesulfonyl) homopiperazine dihydrochloride (informal name: HA-1077), (S)-(+)-2-Methyl-4-glycyl-1-(4-methylisoquinolinyl-5-sulfonyl) homopiperazine dihydrochloride (informal name: glycyl H-1152) and (+)-(R)-trans-4-(1-aminoethyl)-N-(4-pyridyl)cyclohexanecarboxamide dihydrochloride (informal name: Y-27632). The kinase inhibitor can be provided at a concentration sufficiently high that the cells survive and remain attached to the surface. When included in an EC culture medium, the ROCK inhibitor concentration can be about 3 μM to about 10 μM (e.g., about 3, 4, 5, 6, 7, 8, 9, 10 μM Y-27632).

### B. Cells for Compositions

In some embodiments, the human endothelial cells are allogenic. In some embodiments, the human endothelial cells not major histocompatibility complex (MHC) matched. In some embodiments, the human endothelial cells have not been genetically modified to reduce rejection by the immune system such as modifying a cell to express certain cell surface markers such as proteins encoded by either the class II or both the class I and the class II major histocompatibility complex genes.

ECs can be applied to a prepared polymeric substrate to prepare an EC-seeded vascular graft for immediate use, later use, or storage. As used herein, the term “prepared” refers to a polymeric substrate that has been treated in preparation for assembling an EC-seeded vascular graft. “Prepared” encompasses a polymeric substrate that was previously coated (partially or fully) and/or de-gassed. In some embodiments, ECs can be stored, for example in liquid nitrogen tanks, until needed for the treatment of a particular patient. For short-term storage (e.g., about 6-12 months), ECs can be stored at −80° C. or lower (e.g., −80°, −90°, −100°, −110°, −120° C., −130° C., −140° C., −150° C., −160° C., −170° C., −180° C., −190° C., −196° C., or lower). In some embodiments, ECs are maintained at temperature above 0° C. including, without limitation, 4° C., room temperature (about 25° C.), and about 37° C., prior to seeding onto a prepared polymeric substrate. The ability to prepare polymeric vascular grafts comprising “universal” ECs in advance and store them until needed is an important advantage, particularly for treatment of patients with an urgent need. In such embodiments, EC-seeded polymeric vascular grafts are suitable for transplanting onto or implanting into a subject, where the graft induces reduced or no graft rejection in the subject.

In some embodiments, ECs for the vascular grafts provided herein are obtained from cell banks. Generally, cell banks collect cell samples from multiple sources, catalog them according to at least one predetermined characteristic, and store the cells under conditions that keep cells viable. Accordingly, stored or “banked” cells having particular predetermined characteristics are available upon demand.

Preparations comprising EC cells useful for clinical applications must be obtained in accordance with regulations imposed by governmental agencies such as the U.S. Food and Drug Administration. Accordingly, in exemplary embodiments, the methods provided herein are conducted in accordance with Good Manufacturing Practices (GMPs). Good Tissue Practices (GTPs), and Good Laboratory Practices (GLPs).

Reagents comprising animal-derived components are generally not used, and all reagents are purchased from sources that are GMP-compliant. In the context of clinical manufacturing of a cell therapy product, such as in vitro populations of human endothelial cells for vascular grafts as provided herein. GTPs govern donor consent, traceability, and infectious disease screening, whereas the GMP is relevant to the facility, processes, testing, and practices to produce a consistently safe and effective product for human use. See Lu et al., 2009, Stem Cells 27:2126-2135. Where appropriate, oversight of patient protocols by agencies and institutional panels is envisioned to ensure that informed consent is obtained; safety, bioactivity, appropriate dosage, and efficacy of products are studied in phases; results are statistically significant; and ethical guidelines are followed.

In some embodiments, human arterial endothelial cells can be obtained according to the methods described in U.S. Patent Pub. 2016/0244719, incorporated herein by reference in its entirety. AECs obtained according to such methods are characterized by high levels of expression of arterial endothelium markers such as EphrinB2, DLL4, Hey-2, jagged-1, and jagged-2. The AECs are also characterized by low leukocyte adhesion, higher NO production and oxygen consumption, response to shear stress, and capacity to form vascular networks in vitro and in vivo while maintaining expression of arterial markers in such networks. The methods provided herein comprise or consist essentially of culturing mesodermal cells in a serum-free, albumin-free, chemically defined culture medium that is substantially free of insulin and comprises a fibroblast growth factor (FGF), a vascular endothelial growth factor (VEGF), and at least one of a Notch agonist, a TGF-beta inhibitor, and an inhibitor of inositol monophosphatase, where culturing occurs for a length of time sufficient for the cultured mesoderm cells to differentiate into arterial endothelial cells. Amounts of FGF, VEGF, Notch agonist, TGF-beta inhibitor, and inhibitor of inositol monophosphatase useful to differentiate human mesodermal cells (including pluripotent stem cell-derived mesodermal cells) into AECs are described U.S. Patent Pub. 2016/0244719, incorporated herein by reference in its entirety. In some embodiments, the cell culture medium used for AEC differentiation methods described herein comprises each of these components. In other embodiments, the culture medium is substantially free of one or more of these ingredients, the choice of the appropriate provision of said culture media being within the skill of the ordinarily skilled artisan. Culturing can take place on any appropriate surface (e.g., in two-dimensional or three-dimensional culture).

AECs characteristically have the following cell surface biomarker expression profile: CD31⁺/CD144⁺/CD41⁻/CD45⁻. Preferably. AECs express one or more of the following arterial endothelial cell markers: Ephrin B2 (EFNB2). Neuropilin-1 (NRP-1)/CD304. Delta-like 4 (DLL4), and CD184 (cluster of differentiation 184). The Ephrin B2 (EFNB2) gene encodes an EFNB class Ephrin that binds to the EPHB4 and EPHA3 receptors. Neuropilin-1 (NRP1), which is also known as vascular endothelial cell growth factor 165 receptor (VEGF165R), is primarily expressed in arterial endothelial cells. DLL4 is a Notch ligand expressed in arterial endothelial cells (Shutter et al., Genes & Dev. 14:1313-18 (2000)). CD184 is also known as CXCR4 (C-X-C chemokine receptor type 4) or fusin.

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence or measure the level of one or more biological markers of AECs using, for example, via fluorescent in situ hybridization; (FISH; see WO98/45479 published October, 1998, incorporated herein by reference in its entirety), Southern blotting, Northern blotting, or polymerase chain reaction (PCR) techniques, such as qRT-PCR. Quantitative methods for evaluating expression of markers at the protein level in cell populations are also known in the art. For example, flow cytometry is used to determine the fraction of cells in a given cell population that express or do not express biological markers of interest.

Preferably, the AEC population comprises at least 80% arterial endothelial cells. In some embodiments, at least about 80% (e.g., at least 80%, 85%, 90%, 95%, 99%, or more) of cells in the resulting cell population are arterial endothelial cells.

The mesodermal cells can express one or more mesodermal markers including Brachyury (T), EMOS, FOXA2, MIXL1, MSX1, or MSX2. For the methods described herein, mesodermal cells are typically cultured in a culture medium that is free, substantially free, or essentially free of insulin, albumin, or any component derived from a non-human animal (i.e., free of xenogeneic material). As used herein, the term “substantially free” refers to cell culture conditions substantially devoid of a certain component or reagent. Substantially free of insulin means the medium contains less than 1% of original concentration of insulin, or less than 2×10⁻⁵% of insulin by weight, and preferably contains less than 1×10⁻⁵%, less than 0.5×10⁻⁵%, less than 0.2×10⁻⁵% or less than 0.1×10⁻⁵% of insulin.

TGFβ receptor inhibitors appropriate for use in a method of the present invention include, without limitation, SB-431542, SB-525334, A83-01, LY2157299, LY210976, RepSox, SB-505124, D4476, GW788388, SD208, and EW-7197. Preferably, the inhibitor of TGF-beta signaling is SB-431542, a small molecule inhibitor of endogenous Activin and the type I receptor (TGFβ Receptor I) (Inman et al., Mol Pharmacol. 62 (1): 65-74 (2002).

Notch is a single-pass cell-surface receptor that binds to a family of cell-surface ligands including the Delta-like and Jagged families. As used herein, the terms “Notch agonist” and “Notch activator” refer to molecules (e.g., biomolecules, small molecules, chemicals) that bind to Notch receptor and initiate or mediate signaling events associated with Notch activation. Resveratrol (3,4,5-trihydroxystilbene) belongs to a class of polyphenolic compounds called stilbenes and is an activator (agonist) of Notch signaling. Other Notch agonists appropriate for use according to methods for promoting arterial differentiation provided herein include valproic acid and suberoyl bishydroxamic acid. In addition, immobilized or multimerized soluble Notch ligands such as immobilized DLL4 and immobilized Jagged-1 peptide also can be used as Notch activators.

Inositol monophosphatase (IMPase) catalyzes the hydrolysis of myo-inositol monophosphates to myo-inositol, which is required in the phosphoinositide cell signaling pathway. In some embodiments, an inhibitor of IMPase is the biphosphonate L-690,330 ([1-(4-Hydroxyphenoxy)ethylidene]bisphosphonic acid). Lithium also inhibits IMPase to attenuate phosphoinositide signaling (Berridge et al., Cell 59:411-419 (1989)). Other inhibitors of the phosphoinositide signaling pathway include, without limitation, phosphoinositide 3-kinase (PI3K) inhibitor Ly294002, Pictilisib, HS-173, GSK2636771, Duvelisib, TG100-115, GSK1059615, PF-04691502, PIK-93, BGT226, AZD6482, SAR245409, BYL719, CUDC-907, IC-87114, TG100713, Gedatolisib, CH5132799, PKI-402, BAY 80-6946, XL147, PIK-90, PIK-293, PIK-294, Quercetin, Wortmannin, ZSTK474, AS-252424, AS-604850, and Apitolisib.

A suitable working concentration range for chemical inhibitors of IMPase, TGFβ receptors, and other described herein is from about 0.1 μM to about 100 μM, e.g., about 2 μM. 5 μM, 7 μM, 10 μM, 12 μM, 15 μM, 18 μM, or another working concentration of one or more the foregoing chemical inhibitors between about 0.1 μM to about 100 μM.

Preferably, mesodermal cells are cultured in the AEC differentiation medium until at least about 80% (e.g., at least 80%, 85%, 90%, 95%, 98%, or more) of cells in the resulting cell population are arterial endothelial cells.

For several of the biological markers described herein, expression will be low or intermediate in level. While it is commonplace to refer to cells as “positive” or “negative” for a particular marker, actual expression levels are a quantitative trait. The number of molecules on the cell surface can vary by several logs, yet still be characterized as “positive.” Accordingly, characterization of the level of staining permits subtle distinctions between cell populations. Expression levels can be detected or monitored by flow cytometry, where lasers detect the quantitative levels of fluorochrome (which is proportional to the amount of cell surface antigen bound by the antibodies). Flow cytometry or fluorescence-activated cell sorting (FACS) can be used to separate cell populations based on the intensity of antibody staining, as well as other parameters such as cell size and light scatter. Although the absolute level of staining can differ with a particular fluorochrome and antibody preparation, the data can be normalized to a control.

In some embodiments, the arterial endothelial cells are derived from human pluripotent stem cells. As described in U.S. Patent Pub. 2016/0244719, incorporated herein by reference in its entirety, human pluripotent stem cells are cultured for a period of about two days in a serum-free, albumin-free, chemically defined cell culture medium comprising a Bone Morphogenetic Protein (BMP), Activin A, and an activator of Wnt/β-catenin signaling, whereby a cell population comprising mesodermal cells is obtained. The mesodermal cells can express one or more mesodermal markers selected from the group consisting of Brachyury (T), EMOS, FOXA2, MIXL1, MSX1, and MSX2.

Human pluripotent stem cells (hPSCs), either embryonic or induced, provide access to the earliest stages of human development and offer a platform on which to derive a large number of cells for cellular therapy and tissue engineering. Accordingly, in exemplary embodiments, the methods provided herein further comprise differentiating human pluripotent stem cells under conditions that promote differentiation of mesodermal stem cells into arterial endothelial cells. In some embodiments, the method of producing an arterial endothelial cell comprises culturing human pluripotent stem cells in a serum-free, albumin-free, chemically defined culture medium that promotes mesoderm differentiation. Pluripotent stem cell-derived mesodermal cells are then differentiated according to AEC differentiation methods (e.g., those described in U.S. Patent Pub. 2016/0244719, incorporated herein by reference in its entirety), thus producing pluripotent stem cell-derived AECs In exemplary embodiments, the serum-free, albumin-free, chemically defined culture medium that promotes mesoderm differentiation comprises Activin A, Bone Morphogenetic Protein 4 (BMP4), FGF2, and an activator of Wnt/β-catenin signaling. The pluripotent stem cells can be human embryonic stem cells or human induced pluripotent stem cells. As used herein. “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998), incorporated herein by reference in its entirety. These cells express Oct-4, SSEA-3, SSEA-4, TRA-1-60 and TRA-1-81. Pluripotent stem cells appear as compact colonies comprising cells having a high nucleus to cytoplasm ratio and prominent nucleolus. ESCs are commercially available from sources such as WiCell Research Institute (Madison, Wis.).

In some embodiments, the endothelial cells are derived from human induced pluripotent stem cells. For example, for patients without an acute need, induced pluripotent stem cells can derived from the patient to produce patient-specific endothelial cells. As used herein, the term “induced pluripotent stem cells” (“iPS cells”) refers to a pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

Human iPS cells can be used according to a method described herein to obtain ECs having the genetic complement of a particular human subject. For example, it can be advantageous to obtain ECs that exhibit one or more specific phenotypes associated with or resulting from a particular disease or disorder of the particular mammalian subject. In such embodiments, iPS cells are obtained by reprogramming a somatic cell of a particular human subject according to methods known in the art. See, for example, Yu et al., Science 324 (5928): 797-801 (2009); Chen et al., Nat. Methods 8 (5): 424-9 (2011); Ebert et al., Nature 457 (7227): 277-80 (2009): Howden et al., Proc. Natl. Acad. Sci. U.S.A 108 (16): 6537-42 (2011), each of which is incorporated herein by reference in their entirety. Subject-specific somatic cells for reprogramming into iPS cells can be obtained or isolated from a target tissue of interest by biopsy or other tissue sampling methods. In some embodiments, subject-specific cells are manipulated or modified in vitro prior to use. For example, subject-specific cells can be expanded, differentiated, chemically treated, genetically modified, contacted to polypeptides, nucleic acids, or other factors, cryopreserved, or otherwise modified prior to reprogramming and then directed differentiation of the reprogrammed cells to produce subject-specific Ecs.

An important difference between endothelial cells produced from iPS cells from a specific individual and primary endothelial cells isolated from that same individual is that the iPS cell-derived cells are infinitely scalable and are capable of exceeding the Hayflick limit (a certain number of cell divisions). As used herein, the term “Hayflick limit” refers to a finite number of population doublings in vitro before a cell can no longer proliferate and enters senescence (Hayflick L. Exp Cell Res 37:614-36, 1965). While the inherent self-renewal capacity of primary cultured endothelial cells is limited, an almost inexhaustible supply of endothelial cells can be obtained according to the methods provided herein from a single source (e.g., a somatic cell of an individual). Accordingly, in an embodiment of the invention, the ECs are capable of expansion within the tissue culture laboratory such that the numbers of cells obtained is sufficient to treat more than one patient and, in the preferred embodiment, are suitable for cell banking.

Defined medium and substrate conditions for culturing pluripotent stem cells, as used in the methods described herein, are well known in the art. Preferably, the media used herein are chemically defined, albumin-free, and xeno-free. In some embodiments, pluripotent stem cells to be differentiated according to the methods disclosed herein are cultured in a chemically defined, serum-free, albumin-free medium.

In some embodiments, the proportion of endothelial cells in a population of cells is enriched using a cell separation, cell sorting, or other enrichment method, e.g., fluorescence activated cell sorting (FACS), enzyme-linked immunosorbent assay (ELISA), magnetic beads, magnetic activated cell sorting (MACS), laser-targeted ablation of non-endothelial cells, and combinations thereof. Preferably, FACS is used to identify and separate cells based on cell-surface antigen expression. In some embodiments, after obtaining a cell population comprising human Ecs according to a method described herein, the human EC population can be expanded in a culture medium appropriate for proliferating human Ecs including, without limitation, Human Endothelial Serum-Free Medium (Life Technologies, Cat. No. 11111-044), EGM-2 (Lonza, Cat. No. CC-3162), and Endothelial Cell Culture Medium (BD Biosciences, Cat. No. 355054).

### C. Additional Vascular Graft Components

Depending on particular use to which a polymeric vascular graft as described herein will be applied, it will be advantageous in some embodiments for the graft to further comprise one or more bioactive agents. As used herein, the term “bioactive agent” or “active agent” refers to therapeutic, prophylactic, and/or diagnostic agents and includes, without limitation, biologically, physiologically, or pharmacologically active substances that act locally or systemically in the human or animal body. Examples can include, without limitation, small-molecule drugs, peptides, proteins, antibodies, sugars, polysaccharides, nucleotides, oligonucleotides, aptamers, siRNA, nucleic acids, and combinations thereof. “Bioactive agent” includes a single agent or a plurality of bioactive agents including, for example, combinations of two or more bioactive agents.

Bioactive agents appropriate for use with a polymeric graft of this disclosure include, without limitation, pharmaceutical compositions, polypeptides (e.g., chemokines, cytokines), and/or additional therapeutic agents or drugs including, without limitation, anti-thrombogenic agents, anti-proliferative agents, agents that prevent, inhibit, or reduce restenosis or aneurysm formation, antineoplastic/anti-proliferative/anti-mitotic agents, vascular cell growth promoters, vascular cell growth inhibitors, and vasodilating agents. Cytokine and chemokines include, without limitation, interleukin (IL)1α, IL-1β, IL-2, IL-3, IL-4, IL-5, IL-6, IL-7, IL-9, IL-10, IL-12(p40), IL-12(p70), IL-13, IL-15, IL-17, IP-10, eotaxin, interferon γ (IFNγ), granulocyte colony-stimulating factor (G-CSF), granulocyte/macrophage colony-stimulating factor (GM-CSF), macrophage inflammatory protein 1α (MIP-1α), RANTES, tumor necrosis factor-alpha (TNF-α), platelet-derived growth factor (PDGF)-AA, PDGF-AB/BB, TGF-beta, VEGF, and combinations thereof. In some embodiments, the bioactive agent is incorporated into a vascular graft or applied to a vascular graft.

In some embodiments, polymeric vascular grafts comprise one or more additional cell types. For example, smooth muscle cells (SMCs) can be seeded onto a polymeric vascular graft in addition to Ecs. The SMCs can be primary smooth muscle cells or human pluripotent stem cell-derived SMCs. The SMCs can be wild-type, genetically modified, or gene edited.

### II. Methods

The polymeric vascular grafts described herein are useful as arterial or arterial-venous shunts for any vascular or cardiovascular surgical application. Exemplary applications include, without limitation, congenital heart surgery, coronary artery bypass surgery, and peripheral vascular surgery. Accordingly, in another aspect, provided herein are methods of producing and using the polymeric vascular grafts provided herein to treat a blood vessel defect in a subject in need thereof. Such a method can include implanting the polymeric vascular grafts disclosed herein in a subject in need thereof. The terms “individual,” “host,” “subject,” and “patient” are used interchangeably herein. In various embodiments, the polymeric vascular grafts are implanted to replace of a portion of a diseased or damaged blood vessel, for example, to replace a weakened portioned of the aorta or vessels damaged due to trauma or damaged due to a vascular disease.

In some embodiments, a polymeric vascular graft is used to bypass and/or replace a stenotic or partially occluded segment of a blood vessel, for example, in coronary or peripheral artery bypass graft procedure. For example, EC-seeded polymeric vascular grafts of this disclosure are useful for bypass operations in the heart or leg. In another example, EC-seeded polymeric vascular grafts of this disclosure are useful in reconstructive surgeries, for example to correct developmental abnormalities or to repair severe injuries. The vascular grafts are also well suited to provide hemodialysis access in arterial-venous shunts.

In some embodiments, methods of treating as provided herein can comprise performing an anastomosis (i.e., the surgical union of tubular parts) to implant the polymeric vascular graft. Typically, an anastomosis between the in situ artery or vein and the polymeric vascular graft is created by sewing the graft to the in situ vessel with suture. Commonly used suture materials include PROLENE® polypropylene sutures and ePTFE. Accordingly, vascular grafts of this disclosure comprise a suturable material such as PTFE or ePTFE.

One of the major problems with existing autologous venous endothelial cell procedures is that it takes about a month to harvest, grow, seed, and culture the cells on the graft. About 30% of patients cannot undergo the procedure because their medical acute need does not permit waiting for 30 days to obtain an autologous venous endothelial cell graft. Accordingly, this disclosure provides materials and methods that are particularly advantageous over conventional methods. In particular, provided herein are methods in which EC-seeded polymeric vascular grafts are prepared and ready for clinical use within about 10 days. Such grafts are prepared using human ECs produced at scale and frozen until needed. In some embodiments, therefore, the provided methods comprises thawing human Ecs, seeding onto a polymeric substrate, preferably a polymeric substrate that has been at least partially coated with one or more endothelial cell adhesion agents. Preferably, the selected cells are thawed and seeded onto a prepared polymeric substrate, and the EC-seeded polymeric substrate is cultured for fewer than 10 days, and preferably fewer than 7 days (e.g., as few as 2, 3, 4, 5, or 6 days). The cultured polymeric substrate is then delivered to or provided for therapeutic use with the patient within about 10 days, and preferably within about 7 days from the initial request. In embodiments in which AECs are derived from banked iPSC cells according to, for example, AEC differentiation protocols described in U.S. Patent Pub. 2016/0244719, incorporated herein by reference in its entirety, the time from initial request to delivery of a prepared AEC-seeded vascular graft must encompass time to complete the differentiation process. This application provides directed differentiation protocols in which, in some embodiments, human pluripotent stem cells are differentiated into mesodermal cells in about 2-3 days, and the resulting mesodermal cells are induced to differentiate into endothelial cells in approximately 3 days. In such embodiments, seeded vascular grafts can be prepared and ready as an “off-the-shelf” product upon demand. In this case. EC seeded vascular grafts can be provided to a patient in need thereof as soon as they are required. For example, a prepared “universal” EC-seeded vascular graft can be provided using overnight or faster delivery. If produced locally, delivery of a prepared vascular graft can require only a matter of minutes or hours. In some embodiments, prepared “universal” EC-seeded vascular grafts can be purchased and locally stored as cryopreserved, frozen products, in which case EC-seeded grafts can be available for patient use with minimal delay.

Any appropriate method can be used to detect and measure functional and morphological changes following implantation of a polymeric vascular graft of this disclosure. For example, vascular ultrasonography can be performed to evaluate fluid flow in the arteries and veins of the body to detect the presence, severity, and/or specific location of disease. Vascular ultrasonography is a noninvasive ultrasound method (also called duplex ultrasonography) used to examine circulation in the blood vessels of the body. In some embodiments, vascular ultrasonography is used to calculate speed of fluid flow in a blood vessel before and after treatment of the vessel with a polymeric vascular graft as described herein. In some embodiments, contrast-enhanced ultrasonography (CEUS) is used to detect and/or monitor vascular pathologies before and after interventions. Vascular ultrasonography and CEUS are particularly useful to detect and characterize post-intervention restenosis. “Restenosis,” as defined herein, means a narrowing of the lumen of a blood vessel at a previously stenotic site (i.e., the site of balloon inflation during angioplasty), or narrowing of the lumen of a blood vessel or synthetic graft following an interventional procedure (e.g., narrowing of the venous side of an arterial-venous anastomosis following bypass surgery using a graft). Restenosis, as used herein, encompasses occlusion. Restenosis includes any luminal narrowing that occurs following an injury to the vessel wall. Injuries resulting in restenosis can therefore include trauma to an atherosclerotic lesion (as seen with angioplasty), a resection of a lesion (as seen with endarterectomy), an external trauma (e.g., a cross-clamping injury), or a surgical anastomosis.

In another aspect, provided herein are methods for fabricating polymeric vascular grafts. These methods can comprise or consist essentially of coating at least a portion of a polymeric substrate with one or more endothelial attachment agents; and contacting human endothelial cells to the coated polymeric substrate, thereby forming an EC-seeded polymeric vascular graft which is substantially non-adhesive to leukocytes or cellular fragments thereof. As used herein, the term “coating” refers to attaching or depositing, by any suitable process, an endothelial attachment agent of this disclosure onto a polymeric material (e.g., ePTFE) such that the deposited agent covers across some or all surfaces of the material. In some embodiments, coating comprises covering, at least partially, inner lumen surface areas of the polymeric material. Coating of a polymeric material does not have to be complete. In particular, it is preferable in some embodiments to provide composition to only a portion or some portions of the polymeric material to be coated, thus resulting in a polymeric material that is at least partially coated by one or more endothelial attachment agents. In some embodiments, a coating includes one or more coating layers. A coating can have a substantially constant or a varied thickness.

In some embodiments, coating at least a portion of the polymeric substrate is performed at room temperature or at a temperature that is physiologically relevant to endothelial cells such as 37° C. In some embodiments, coating comprises contacting at least a portion of the polymeric substrate with one or more endothelial attachment agents for any appropriate length of time including, without limitation, a few minutes, a few hours, or about 12 hours to about 24 hours, whereby a partially or fully coated substrate is obtained.

In some embodiments, the method optionally comprises de-gassing the polymeric substrate prior to coating with one or more endothelial cell attachment agents.

In another aspect, provided herein are methods for cryopreserving EC-seeded polymeric vascular grafts. Cryopreservation is a process wherein biological materials such as cells, tissues, extracellular matrix, organs, or any other biological constructs susceptible to damage caused by unregulated chemical kinetics are preserved by cooling to very low temperatures (typically −40° C. or −80° C.). The method can comprise or consist essentially of contacting an EC-seeded polymeric vascular graft to a cryoprotectant (also referred to as cryoprotective agents, cryoprotectant agents, and cryopreservatives) and then exposing the contacted material to freezing temperatures. The cryoprotectant protects biological material on the vascular graft from the damaging effects of freezing (such as ice crystal formation and increased solute concentration as the water molecules in the biological material freeze). In some embodiments, the cryopreserved vascular graft retains at least 40%, 45%, 50%, 55%, 60%, 65%, 70%, 75%, 80%, 85%, 90% or 95% EC cell viability after freezing and thawing as determined by the cell count on the graft tissue before processing and cell count in the graft after freezing and thawing.

In another aspect, provided herein are methods for delivering an endothelial cell-seeded vascular graft to a patient in need thereof, the methods comprising seeding human endothelial cells onto a polymeric substrate at least partially coated by an endothelial cell attachment agent: culturing the seeded polymeric substrate for about 2 to about 10 days, whereby an EC-seeded polymeric substrate suitable for implantation as a vascular graft is produced; and providing the EC-seeded vascular graft within about 10 days from when the graft is needed by the patient. In some embodiments, the vascular graft can be cryopreserved for a long-term storage. The polymeric substrate can be expanded polytetrafluoroethylene (ePTFE), poly vinyl chloride (PVC), PGA (poly glycolic acid), PLA (poly lactic acid), PCL (poly caprolactone), PGLA (polylactic-co-glycolic acid), polyurethane, polydioxanone, polyethylene, polyethylene terephthalate (Dacron®), tetrafluoroethylene (TFE), polytetrafluoroethylene (PTFE), silk, decellularized scaffold, an extracelluar matrix protein-based scaffold, hyaluronic acid, chitosan, and polyhydroxyalkanoate. The endothelial cell attachment agent can comprise one or more of dopamine, fibrin glue, RGD peptides, vitronectin, or laminin, or appropriate combinations thereof.

In some embodiments, the vascular graft exhibits reduced leukocyte adhesion relative to a polymeric substrate seeded with venous endothelial cells. In some embodiments, the vascular graft exhibits reduced thrombosis relative to a polymeric substrate seeded with venous endothelial cells or a naked, uncoated polymeric substrate. In some embodiments, the vascular graft exhibits increased long-term patency rates relative to a polymeric substrate not coated with the endothelial cell attachment agent. In some embodiments, the method further comprises de-gassing the polymeric substrate prior to coating with the endothelial cell attachment agent. In some embodiments, de-gassing comprises washing the polymeric substrate in acetone and ethanol, washing the polymeric substrate in an organic solvent, or applying a vacuum. In some embodiments, the human endothelial cells are allogenic. In some embodiments, the human endothelial cells are non-major histocompatibility complex matched. In some embodiments, the human endothelial cells have not been genetically modified to reduce rejection by the immune system.

In another aspect, provided herein are methods for delivering endothelial cell-seeded vascular grafts, the methods comprising: upon receipt of a request for an endothelial cell-seeded vascular graft, selecting a cryopreserved endothelial cell (EC)-seeded vascular graft suitable for a subject in need thereof: thawing the selected cryopreserved EC-seeded vascular graft: removing cryopreservation solution from the thawed EC-seeded vascular graft, if present; and delivering the EC-seeded vascular graft within about 1-2 days (e.g., within about 24 to about 48 hours) from receipt of the request. Importantly, these methods provide a solution to a critical need for patient care, specifically the ability to provide a patient-ready EC-seeded vascular graft within one to two days (e.g., within about 24 to about 48 hours) of receipt of a request for the graft material. These methods thus provide a significant improvement over conventional methods, which require about 30 days to provide a vascular graft seeded with the patient's autologous venous endothelial cells. As used herein, the term “patient-ready” means that the graft is pre-configured and is ready for use with a patient with minimal delay or additional preparation.

### III. Articles of Manufacture

In another aspect, provided herein are articles of manufacture. For example, provided herein is a container comprising a cryopreserved combination product and a cryopreservation solution, wherein the cryopreserved combination product comprises a human arterial endothelial cell population seeded onto an implantable polymeric substrate at least partially coated by an endothelial cell attachment agent. In some embodiments, the endothelial cell attachment agent comprises dopamine. In some embodiments, the human endothelial cells are allogeneic. In some embodiments, the human endothelial cells are not major histocompatibility complex (MHC) matched. In some embodiments, the human endothelial cells have not been genetically modified to reduce rejection by the immune system such as modifying a cell to express certain cell surface markers such as proteins encoded by either the class II or both the class I and the class II major histocompatibility complex genes.

The container can be a vial, cryotube, bag, or any other vessel suitable to contain a polymeric vascular graft and a cryopreservation solution. Preferably, the container can be stored at freezing temperatures including, without limitation, a temperature from 1° C. to about −196° C. or lower (e.g., 1°, 0°, −1°, −5, −10°, −20°, −30°, −40°, −50°, −60°, −70°, −80°, −90°, −100°, −110°, −120°, −130°, −140°, −150°, −160°, −170°, −180°, −190°, −196° C., or lower).

In some embodiments, the human endothelial cell population is contacted with a cryopreservation solution prior to seeding onto the implantable polymeric substrate. In other embodiments, the implantable polymeric substrate is contacted to a cryopreservation solution after seeding by human endothelial cells. Examples of suitable cryopreservation solutions include, without limitation, dimethyl sulfoxide (DMSO). In some embodiments, a 10% DMSO solution is used for cryopreservation. In some embodiments, the cryopreservation solution is removed from the seeded implantable polymeric substrate prior to implantation. The solution contacting and removal steps are generally carried out under aseptic, preferably sterile, conditions.

In another aspect, provided herein is a container comprising a cryopreserved combination product and a cryopreservation solution, wherein the cryopreserved combination product comprises a human endothelial cell-seeded implantable polymeric substrate, wherein the implantable polymeric substrate is at least partially coated by one or more endothelial cell attachment agents. The polymeric substrate can be selected from expanded poly tetrafluoroethylene (ePTFE), poly vinyl chloride (PVC), PGA (poly glycolic acid), PLA (poly lactic acid), PCL (poly caprolactone), PGLA (polylactic-co-glycolic acid), polyurethane, polydioxanone, polyethylene, polyethylene terephthalate (Dacron®), tetrafluoroethylene (TFE), polytetrafluoroethylene (PTFE), silk, decellularized scaffold, an extracelluar matrix protein-based scaffold, hyaluronic acid, chitosan, and polyhydroxyalkanoate. In some embodiments, the human endothelial cells are non-immunogenic to a recipient of the implantable polymeric substrate. The cryopreservation solution can comprise about 10% dimethyl sulfoxide (DMSO). In some embodiments, the human endothelial cell population is contacted with cryopreservation solution prior to seeding onto the implantable polymeric substrate. Preferably, the cryopreservation solution is removed from the seeded implantable polymeric substrate prior to implantation. Preferably, the combination product is configured for storage at a temperature from 37° C. to about −196° C. (e.g., about 37°, 30°, 25°, 15°, 10°, 4°, 1°, 0°, −1°, −5, −10°, −20°, −30°, −40°, −50°, −60°, −70°, −80°, −90°, −100°, −110°, −120°, −130°, −140°, −150°, −160°, −170°, −180°, −190°, −196° C., or lower) without a significant loss of cell viability relative to a control not stored under such conditions.

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application are specifically and individually indicated to be incorporated by reference, unless only specific sections of patents or patent publications are indicated to be incorporated by reference.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following examples are provided to better explain the various embodiments and should not be interpreted in any way to limit the scope of the present disclosure.

### Example 1: Generation of Vascular Graft

Fibrin glue coating: Fibrinogen component (Baxter, TISSEEL) was prepared by diluting a 2 ml portion of Fibrinogen with 4 ml heated Fibrinolysis inhibitor, and then adding 1 ml Tranexamic acid (20 mg/ml). Thrombin component was prepared by diluting 2 ml Fibrinogen in 4 ml CaCl₂, 75 ml H₂O, and 4 ml Tranexamic acid (20 mg/ml). Fibrinogen component was flowed through the ePTFE three times. Next, thrombin component was flowed through the ePTFE for 5 minutes. The ePTFE was rinsed with distilled water 3 times. After the coating steps were repeated once, ePTFE was flushed with 5 ml 50 U/ml heparin.

Dopamine coating: Dopamine was dissolved into 10 mM Tris solution (pH=8.5) at 2 mg/mL concentration. ePTFE was immersed into the solution immediately and incubated in the solution at room temperature or 37° C. for 4-24 hours. Coated ePTFE was washed five times with distilled water.

Seeding cells on ePTFE: Endothelial cells were suspended at a density of (1.5×10⁶ cells/ml) in cell culture medium comprising Y27632 (a ROCK inhibitor) and seeded onto the ePTFE. The ePTFE was put into a tube and then loaded into a cell-seeding device (Endostradilisator III, Biggler). The ePTFE (in the tube) was rotated for 3 hours at 4 rph. Alternatively, the ePTFE can be incubated for 1 hour, then manually turned 90° and incubated for another hour. The 90° rotation was repeated for 4 times.

De-gas of ePTFE: De-gassing was performed by immersing ePTFE in acetone for 3 hours and then washing the acetone-treated ePTFE with 70% Ethanol for 30 minutes (repeated 3 times). The de-gassed ePTFE was rinsed in distilled water for 30 minutes (repeated 3 times). From this time point, ePTFE needs to be immersed in distilled water or phosphate buffered saline (PBS) to avoid re-gas.

### Example 2: Implantation of Vascular Graft

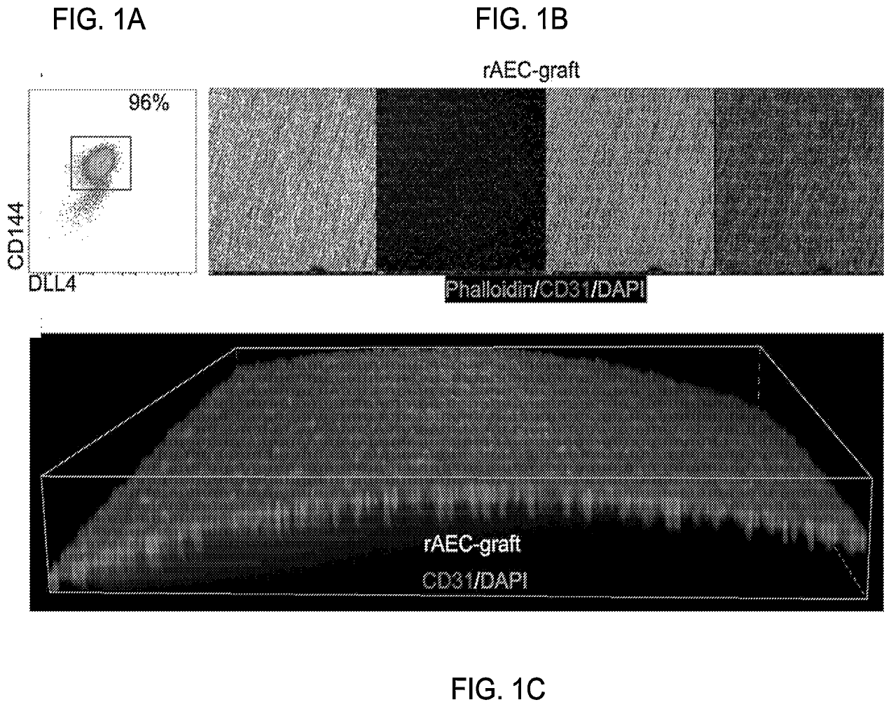

A small diameter vascular graft seeded with allogeneic endothelial cells (3 mm diameter) was generated according to the methods described in Example 1 and such as those described by U.S. patent application Ser. No. 16/556,674, incorporated herein by reference as if set forth in its entirety. (AEC-seeded graft) (FIGS. 1B & 1C). The arterial endothelial cells were generated from rhesus embryonic stem cells (FIG. 1A).

The AEC seeded- and a naked-vascular graft (not seeded with any AECs) were transplanted in the femoral artery of a rhesus monkey model animal. The naked vascular graft started to fail as early as 2 weeks post transplantation and 50% of the naked grafts failed within 6 months. Further experiments demonstrated that the naked grafts failed due to thrombosis. Surprisingly, the AEC-seeded vascular grafts prevented thrombus and maintained patency for at least 6 months. It was shown that the allogenic donor AECs were destroyed by the host immune system over time and replaced by host endothelial cells, which eliminated the tumorigenesis risk.

Additionally, allogenic AEC-seeded grafts were compared to grafts seeded with double knockout AECs, wherein the AECs had a B2M and CIITA double knockout (DKO-AEC graft). The patency of DKO-AEC-graft was similar to the patency of the naked-graft.

Repopulation of Vascular Grafts with Endogenous Endothelial Cells

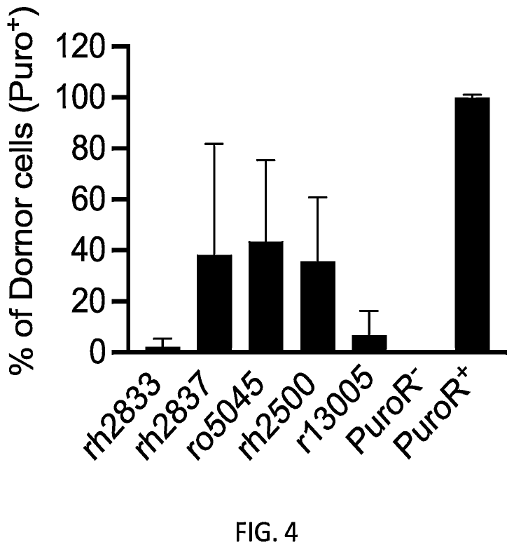

Analysis of vascular graft produced and employed as set forth herein revealed that within six months of insertion a substantial percentage of the cells in the vascular grafts had been repopulated by host cells or were a mosaic of donor and host endothelial cells. These results are shown in FIG. 4. In order to ascertain whether these endothelial cells were repopulated by host (PuroR−) cells, isolated genomic DNA was obtained from endothelial cells that were sorted from the MHC-WT grafts six months post implantation. qPCR analysis of a puromycin resistance gene revealed that 2-40% endothelial cells were PuroR⁺, indicating the rest of endothelial cells (60-98%) were from the host (PuroR⁻).

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

We claim:

1. A method of treating a blood vessel defect in a subject in need thereof comprising implanting a vascular graft in a subject, wherein the vascular graft comprises (a) a polymeric substrate at least partially coated by an endothelial cell attachment agent and (b) human endothelial cells adhered to said coated polymeric substrate.

2. The method of claim 1, wherein the vascular graft is implanted to replace a portion of a diseased or damaged blood vessel.

3. The method of claim 2, wherein the weakened portioned vessel is a result of trauma.

4. The method of claim 2, wherein the weakened portioned vessel is a result of vascular disease.

5. The method of claim 1, wherein the vascular graft is implanted by anastomosis.

6. The method of claim 1, wherein the endothelial cells are not autologous cells.

7. The method of claim 1, wherein the human endothelial cells are allogeneic cells.

8. The method of claim 7, wherein the human endothelial cells are not major histocompatibility complex (MHC) matched to the subject.

9. The method of claim 8, wherein the human endothelial cells have not been genetically modified to reduce rejection by the immune system.

10. The method of claim 1, wherein the endothelial cells are arterial endothelial cells.

11. The method of claim 1, wherein the endothelial cells are venous endothelial cells.

12. A method of implanting a vascular graft in a subject in need thereof, wherein the vascular graft comprises (a) a polymeric substrate at least partially coated by an endothelial cell attachment agent and (b) human endothelial cells adhered to said coated, polymeric substrate.

13. The method of claim 12, wherein the vascular graft is implanted by anastomosis.

14. The method of claim 12, wherein the endothelial cells are not autologous cells.

15. The method of claim 12, wherein the human endothelial cells are allogeneic cells.

16. The method of claim 15, wherein the human endothelial cells are not major histocompatibility complex (MHC) matched to the subject.

17. The method of claim 16, wherein the human endothelial cells have not been genetically modified to reduce rejection by the immune system.

18. The method of claim 12, wherein the endothelial cells are arterial endothelial cells.

19. The method of claim 12, wherein the endothelial cells are venous endothelial cells.

20. The method of claim 12, wherein the vascular graft is implanted during congenital heart surgery, coronary artery bypass surgery, or peripheral vascular surgery.

21. A vascular graft comprising (a) a polymeric substrate at least partially coated by an endothelial cell attachment agent and (b) human endothelial cells adhered to said coated polymeric substrate.

22. The vascular graft of claim 21, wherein the endothelial cells are not autologous cells.

23. The vascular graft of claim 21, wherein the human endothelial cells are allogeneic cells.

24. The vascular graft of claim 23, wherein the human endothelial cells are not major histocompatibility complex (MHC) matched to the subject.

25. The vascular graft of claim 21, wherein the human endothelial cells have not been genetically modified to reduce rejection by the immune system.

26. The vascular graft of claim 21, wherein the endothelial cells are arterial endothelial cells.

27. The vascular graft of claim 21, wherein the endothelial cells are venous endothelial cells.
